(19) United States
(12) Patent Application Publication
(10) Pub. No.: US 2026/0233187 A1
(43) Pub. Date: Aug. 13, 2026

(54) COMPARTMENTED REACTION VESSEL, SYSTEM AND METHOD FOR CONVERTING A SUBSTRATE TO A PRODUCT

(71) Applicant: USW Commercial Services Ltd, Pontypridd, Wales (GB)

(72) Inventors: Sandra ESTEVES, Pontypridd (GB); Timothy PATTERSON, Pontypridd (GB); Savvas SAVVAS, Pontypridd (GB)

(21) Appl. No.: 19/151,586

(22) PCT Filed: Feb. 29, 2024
(86) PCT No.: PCT/EP2024/055188

§ 371 Date: Jul. 28, 2025

(30) Foreign Application Priority Data
Mar. 1, 2023 (GR) 20230100173
Mar. 7, 2023 (GB) 2303326.9

(51) Int. Cl. B01J 19/00; B01J 19/18; B01J 19/30; C12M 1/00; C12M 1/107; C12M 1/34; C12M 3/04; C12P 5/02

(52) U.S. Cl. CPC B01J 19/006; B01J 19/1856; B01J 19/1862; B01J 19/305; C12M 21/04; C12M 23/34; C12M 23/58; C12M 27/10; C12M 27/20; C12M 41/26; C12M 41/44; C12P 5/023; B01J 2219/0077; B01J 2219/00772

(57) ABSTRACT

The present invention provides a reaction vessel for converting a substrate to a product, comprising an inlet opening for introducing a liquid medium and/or a substrate within said reaction vessel, an outlet opening for obtaining a product from said reaction vessel, a length direction extending substantially along a central axis of said reaction vessel, and a plurality of baffles distributed along a circumference of an inner surface of the reaction vessel, wherein each baffle extends along said length direction and from an inner surface of the reaction vessel towards an interior of the reaction vessel, wherein, for each baffle, the reaction vessel is rotatable around the central axis into a position in which the baffle extends from a bottom half of the inner surface at least to a horizontal plane coinciding with the central axis, thereby dividing a bottom half of the internal volume of the reaction vessel into compartments.

### TECHNICAL FIELD

The present invention relates to the conversion of substrates to products, more particularly to a reaction vessel for converting a substrate(s) to a product(s), and a system comprising such a reaction vessel, and further to methods applying such reaction vessels and systems for converting a substrate(s) to a product(s).

### BACKGROUND

Conversion of substrates such as CO₂, CH₄, CO, H₂ and N₂ to various products, by chemical conversion as well as by biological conversion, are significant areas of research and development and the scale-up of such technologies are of particular interest in a circular, low carbon economy. Numerous gases have a low solubility in water and therefore gas-liquid mass transfer typically requires some form of high intensity mixing. Achieving process stability, high product efficiencies and low energy input is therefore critical.

In the last decade, an increasing interest in the process of hydrogenotrophic methanogenesis brought biofilm reactors into attention as an approach to reduce the parasitic energy losses associated with more conventional reactors (Patterson et al., 2017; Savvas et al., 2017). Hydrogenotrophic methanogenesis is a biological reaction executed by a group of lithotrophic archaea and involves the production of CH₄ from H₂ and CO₂. H₂ and CO₂ can also be converted to CH₄ via homoacetogenesis followed by acetoclastic methanogenesis. The bioconversion can only take place after diffusion of the feed gases (H₂ and CO₂) into the liquid media surrounding the microbes and typically this is achieved by means of intense gas/liquid agitation. The energy losses from agitation depend on the mixing method and intensity but are calculated to be substantial (Gill et al., 2008; Karimi et al., 2013). Furthermore, with regards to commercialisation, high intensity mixing brings issues with reactor scalability, as for the same gas mass transfer rates, the shear forces imposed to the culture increase with an increase in reactor size, causing physical damage to microbial cells (Garcia-Ochoa and Gomez, 2009, 2004).

With regards to biofilm reactor architecture, the majority of biomethanation biofilm reactor studies are currently based on the trickling bed (TB) reactor design (e.g., Strübing et al., 2018). Typically, TB biomethanation reactors employ a counter-current flow of liquid media and gas over a fixed bed that serves as microbial attachment media. Biofilm forms on the fixed bed and acts as a catalyst for the conversion of H₂ and CO₂ to CH₄. The biofilm is never fully immersed in the liquid media, and this is considered to offer significantly higher surface area of contact between the gas and the liquid phase as compared to other biofilm reactor types. Yet, several issues need to be addressed for the biofilm technology to reach higher levels of adoption in the emerging biological gas conversion industry. The main issues are reaction rates, liquid media distribution, and nutrient availability. Biomethanation biofilm reactors have yet to reach the reaction rates achieved by systems that rely on intensive mixing (Rusmanis et al., 2019). Concurrent or counter-current gas-liquid flow over a packed bed is famously difficult to predict and trickling beds almost always exhibit partial solid wetting (Mocciaro et al., 2011). In the case of biomethanation, dry areas are detrimental to biofilm formation and will unavoidably lead to diminished biomethane production. Equally important, uneven liquid media distribution leads to uneven nutrient distribution, with expected oligotrophic and eutrophic areas and the effect of channelling of both liquid and gas (Wik, 2003), all of which acts to reduce reactor efficiency. A matter of further concern is the production of water, a methanogenesis metabolite. In TB reactors, metabolically produced water from the upper regions is travelling together with the nutrient media to the bottom of the reactor, increasingly diluting nutrient concentration. At lab scale the effects of this phenomenon might be minimal over experimental timeframes but in commercial size reactors and operations it is expected to rapidly reduce performance. Further, in biological or chemical conversion of other gaseous substrates as well as non-gaseous substrates, there is also room for improvement of issues like reaction rates, liquid media distribution, and nutrient availability.

### SUMMARY

The object of the present invention is to solve or at least mitigate the above-mentioned issues associated with previously known reaction vessels. This is achieved by providing a compartmented reaction vessel, which requires a volume of liquid medium smaller than half of the internal volume of the reaction vessel to wet the entirety of the internal volume of the reaction vessel upon rotation of the reaction vessel.

More particularly, according to a first aspect of the present invention, there is provided a reaction vessel for converting a substrate to a product, comprising:
an inlet opening for introducing a liquid medium and/or a substrate within said reaction vessel,
an outlet opening for obtaining a product from said reaction vessel,
a length direction extending substantially along a central axis of said reaction vessel, and a plurality of baffles distributed along a circumference of an inner surface of the reaction vessel, wherein each baffle extends along said length direction and from an inner surface of the reaction vessel towards an interior of the reaction vessel,
wherein, for each baffle, the reaction vessel is rotatable around the central axis into a position in which the baffle extends from a bottom half of the inner surface at least to a horizontal plane coinciding with the central axis, thereby dividing a bottom half of the internal volume of the reaction vessel into compartments,
wherein the plurality of baffles are configured to allow a liquid medium contained within the reaction vessel to move sequentially from a first one of the compartments into a neighbouring one of the compartments during rotation of the reaction vessel.

The function of the baffles is to minimise the volume of liquid medium that is needed to wet the entire interior volume of the reaction vessel.

According to a second aspect of the present invention, there is provided a system for converting a substrate to a product, comprising:
a reaction vessel, as disclosed in detail elsewhere herein, and
a rotational device for rotating said reaction vessel around the central axis of the reaction vessel.

According to a third aspect of the present invention, there is provided a method for converting a substrate to a product, comprising operating a reaction vessel, as disclosed in detail elsewhere herein, or a system, as disclosed in detail elsewhere herein. More particularly, the method comprises adding a liquid medium and a substrate to the reaction vessel via the inlet of the reaction vessel, applying a rotational movement of the reaction vessel to allow wetting of the entire interior volume of the reaction vessel by the liquid medium, and obtaining a product from an outlet of the reaction vessel, wherein the volume of liquid medium contained within the reaction vessel is smaller than half of the interior volume of the reaction vessel at each moment of converting the substrate to the product within the reaction vessel.

Further features and advantages of the invention will become apparent from the following description of preferred embodiments of the invention, given by way of example only, which is made with reference to the accompanying drawings.

### BRIEF DESCRIPTION OF THE DRAWINGS

FIG. 1a shows a side view of a reaction vessel according to an embodiment.

FIG. 1b shows a cross-sectional, perspective view of a portion of a reaction vessel according to an embodiment.

FIGS. 2a and 2b show cross sections of reaction vessels according to some embodiments.

FIGS. 3a to 3d show cross sections of reaction vessels according to further embodiments.

FIGS. 4a and 4b are schematic outlines of systems for converting a substrate to a product according to some embodiments of the present invention.

FIG. 5 schematically depicts a method for converting a substrate to a product by operating a reaction vessel or a system as disclosed herein.

FIGS. 6a and 6b shows the evolution in methane production from start-up to steady state in a Rotating Compartmented Biofilm Reactor System (RCBRS) according to the present invention and a previously known Continuously Stirred Tank Reactor (CSTR), respectively.

FIG. 7 shows how methane formation rate increases/decreases relative to rotational speed of the reaction vessel according to the present invention.

FIG. 8 shows the effect of gas input rate and impeller speed on methane formation in a previously known CSTR.

FIG. 9 shows the effect of change of gas input rate, ratio, and pressure on methane production efficiency in a reaction vessel according to the present invention.

FIG. 10 shows conversion to methane from H₂ and CO₂ in the RCBRS according to the present invention.

FIG. 11 demonstrates conversion to methane from H₂ and CO₂ in the RCBRS at higher inflow of gas.

FIG. 12 shows the effect of pressure on conversion efficiency in the RCBRS.

FIG. 13 illustrates the difference in microbial culture intermediate metabolites produced and accumulated on a CSTR and on the RCBRS after one month of operation, respectively.

### DETAILED DESCRIPTION

The present invention is directed to a reaction vessel for converting a substrate to a product. The reaction vessel is suitable for use in a biological conversion as well as a chemical conversion, of a substrate which may be gaseous or non-gaseous (i.e., liquid, or solid) or a combination of substrates, to a product which is gaseous or non-gaseous or a combination of products. A system comprising the herein disclosed reaction vessel is also disclosed, as well as a method for converting a substrate to a product.

All words and terms used throughout the present text shall be considered to have the same meaning usually given to them by the person skilled in the art unless another meaning is apparent from the context.

Herein, the term ‘reaction vessel’ is defined to include a container, receptacle, repository such as a tank, which is suitable for performing reactions of converting a substrate to a product.

The terms ‘internal volume of a reaction vessel’ and ‘interior volume of a reaction vessel’ are intended to mean the total inner volume of a reaction vessel and these terms may be used interchangeably herein, whereas the term ‘free volume of a reaction vessel’ is intended to mean the inner space not occupied by baffles, any other internal part of the reaction vessel, and any packing material, if present in the reaction vessel.

Further, the term ‘packing material’ is intended to mean a support frame that is evenly distributed within the free volume of a reaction vessel in packed bed reactors, either across the whole length of the reaction vessel, or only across part(s) of the length of the reaction vessel. A non-limiting embodiment includes alternating packed and non-packed parts along the length of the reaction vessel. Herein, the term ‘packing material’ may be used interchangeably with the terms ‘support frame’, ‘solid phase’, ‘support (porous) medium/media’, and ‘packing medium/media’. In an embodiment, the packing material may act as a ‘microbial attachment device’ or, in short, an ‘attachment medium/media’.

In the present context, the term ‘substrate’ is intended to mean a starting material, compound, or substance, which through a biological, biochemical, or chemical reaction, can be converted into one or more products, and/or intermediate products. The term ‘product’ means the final (or end) material, compound, or substance, which is the result of a biological, biochemical, or chemical reaction or process. The term ‘intermediate product’ is construed as meaning the product of a first biological, biochemical, or chemical reaction, which intermediate product is subsequently used as the substrate for a second biological, biochemical, or chemical reaction, in which reaction, the substrate is converted into a desired, second product.

Herein, the term ‘a plurality of baffles’ means any number of baffles higher than 1 (i.e., >1), that is, at least 2 baffles (i.e., ≥2), as further described in more detail elsewhere herein.

Herein, a reaction vessel or system ‘comprising’ one or more recited elements, may also include other elements not specifically recited. Similarly, a method or process ‘comprising’ one or more recited steps may also include other steps not specifically recited.

Further, the singular ‘a’ and ‘an’ shall be construed as also including the plural.

Herein, it is shown that the presently disclosed reaction vessel is able to significantly improve biological gas conversions by:
a. Enhancing gas-liquid mass transfer rates but at the same time reducing foam generation
b. Improving liquid distribution and nutrient availability
c. Reducing parasitic energy losses
d. Improving ease of scalability

Parameters a and b are linked to the same principle of reducing the liquid phase inside the reactor to the essential minimum. In order to grow and thrive, the microbes responsible for the conversion need to be surrounded by a water layer that also carries the ions and macromolecules that are essential for their metabolism. Whether attached on a surface or in suspension, when the culture is completely submerged in a solution, the gaseous substrate will be in the form of bubbles. In this case, the rate of gas-liquid mass transfer increases by the reduction in bubble size which accomplishes the increase of the surface area of contact between the gas and the liquid phase and the reduction of the buoyancy force exerted on the bubbles. The reduction in bubble size to an effective level is conventionally achieved by intense gas-liquid mixing or other means of mechanical agitation which increases the energy input to the system.

Packed biofilm reactors allow the microbes to attach to a support frame (i.e., a packing material) that is evenly distributed across the whole internal volume of the reaction vessel and by eliminating most of the liquid inside the vessel, the gas molecules reach the biofilm by diffusion through space and are not restricted in packets as in the case of bubbles. The baffles located in the reaction vessel according to the present invention achieves a reduction in the thickness of the liquid layer that surrounds the microbes, which means that the path travelled by the gas to reach the microbes is shortened. This results in the microbial gas uptake to occur closer to the point of diffusion thus increasing the concentration gradient between the gas and the liquid phase. This increases the gas-liquid mass transfer rate as shown by equation 1.

$$dq / dt = {k}_{L} α ( {C}_{g} - {C}_{l} ) \qquad ( Eq . 1 )$$

Where,
k_{L} is the linear mass transfer coefficient,
α is the specific surface area of contact between the gas and the liquid and
(C_{g}−Cₗ) is the concentration gradient between the gas phase and the liquid phase.

Additionally, in the proposed design, diffusion is achieved without energy expenditure for intense agitation which has a drastic positive effect on the net energy gain of the system.

Enrichment of the reactor takes place by inoculation of the liquid media. Gas flows are dictated by conversion rates and biofilm formation time.

It is expected that the herein disclosed reaction vessel and system will provide improved conversion efficiency not only for biological conversions, such as in a biofilm reactor, but also for chemical conversion of substrates to products, thanks to the following factors:
It allows for the increase of surface area of contact between the gas and the liquid phase and further allows for the adjustment of the thickness of the liquid layer within the reaction vessel. This allows for adjustment of the rate at which gases and/or liquids reach any microbial population and/or chemical catalyst or reaction site contained within the vessel. The adjustment is achieved through the arrangement of baffles in the vessel, and additionally through the regulation of the rotational speed of the vessel.
It attains a uniform distribution of nutrients, water, gases, and liquids for an entire microbial population and/or chemical catalyst well distributed within the reaction vessel. This is achieved through the complete but only periodical immersion of the microbial population and/or chemical catalyst/reactants/additives by liquid medium.
The energy expenditure for the rotation of the vessel is minimal and not proportional to the gas conversion rate.
The reaction vessel is not limited by channelling or by foam and gas entrapment
The reaction vessel allows an effective gas product disengagement.

The different aspects of the present invention will now be described in detail by reference to the drawings.

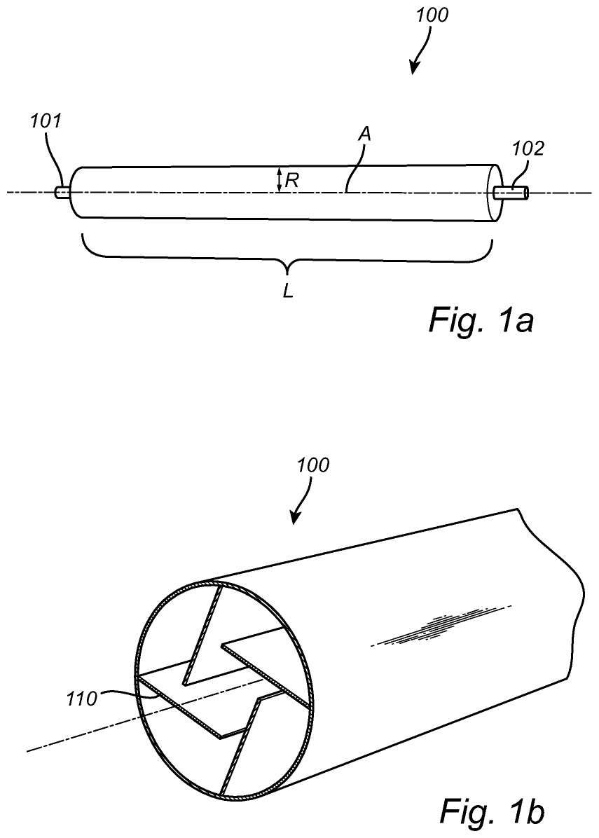

FIG. 1a is a schematic side view of a reaction vessel 100 according to an embodiment of the present invention. The reaction vessel 100 has a main direction of extension, also referred to as a length direction, oriented along a central axis A, as well as an inlet opening 101 and an outlet opening 102. The inlet opening 101 may be employed to supply the substrate to the interior of the vessel 100, and the outlet opening 102 to discharge the resulting intermediate product or product from the vessel 100. Further, any unconverted substrate would also be discharged via the outlet opening 102. In the present example, the inlet opening 101 and the outlet opening 102 are arranged at opposite end portions of the vessel 100, with respect to the central axis A. However, it is to be understood that other arrangements of the inlet and the outlet are possible, provided that they allow unrestricted flow of components to and from the reaction vessel and allow keeping a liquid level within the reaction vessel as close to the central axis as possible, or alternatively, at or below the central axis.

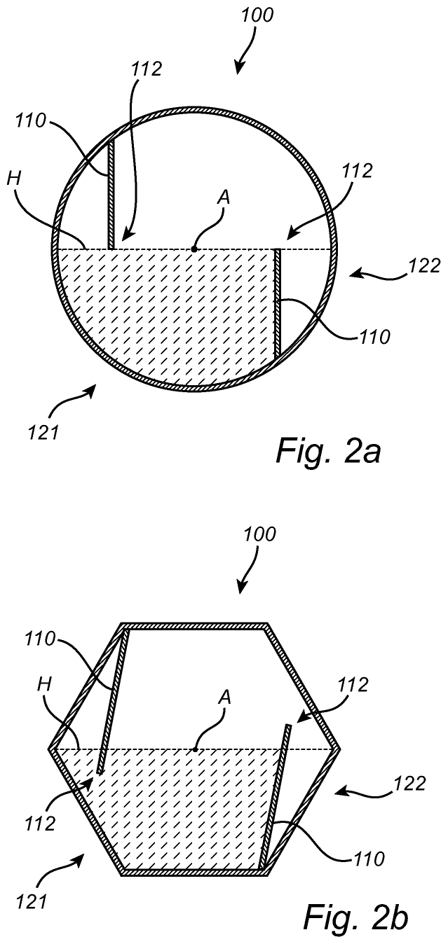

It will be appreciated that the main body of the reaction vessel 100, defining its interior volume, may be rotationally symmetric around the central axis A. In the present example illustrated in FIG. 1a, the reaction vessel has a main body conforming to a right circular cylinder with a radius R, length L and a cylinder axis coinciding with the central axis A. Other shapes are however possible, as will be discussed later in connection with, for example, FIG. 2b

FIG. 1b is a cross-sectional perspective view of the reaction vessel 100 of FIG. 1a, in which the cross section is taken through a plane orthogonal to the central axis A. The cross section illustrates a plurality of baffles 110, or interior walls, dividing the internal volume of the reaction vessel 100 into compartments or chambers. The baffles 110 are distributed along the circumference of the inner surface of the reaction vessel 100, and oriented to extend from the inner surface towards an interior of the reaction vessel 100. Further, each baffle 110 has a length (not shown in the present figure) which preferably may extend along the entire length L of the reaction vessel 100. The embodiment depicted in FIG. 1b comprises four baffles 110. However, other configurations are also possible, comprising for example two, three, five or more baffles 110. Preferably, the baffles 110 are evenly distributed within the reaction vessel 110 to promote rotational symmetry and substantially equally sized compartments or chambers.

Each baffle 110 may be shaped as a blade or wall that is sealed to the inner surface of the reaction vessel 100 to provide a barrier for any liquid contained in the vessel 100. Hence, the barrier formed by a baffle 110 may cause the liquid to flow over the top edge of the baffle 110 (i.e., the edge facing the central axis A) as the vessel 100 rotates around the central axis A.

FIG. 2a shows a cross section of a reaction vessel 100, taken orthogonally to the central axis A. The reaction vessel 100 may be similarly configured as the reaction vessels 100 disclosed in FIGS. 1a and b. In the present example, the reaction vessel 100 comprises a first and a second baffle 110 extending from the inner surface of the reaction vessel 100 towards the interior of the reaction vessel 100. As shown in the present cross section, the baffles 110 may be evenly distributed within the vessel 100, allowing for a two-fold rotational symmetry as the reaction vessel 100 rotates around the central axis A. Thus, rotating the reaction vessel 180° would result in a similar orientation of the baffles 110 as the one shown in FIG. 2a.

As mentioned above, each of the baffles 110 may be sealed to the inner surface of the reaction vessel 100 and extend towards the interior of the reaction vessel 100. As the reaction vessel 100 rotates around the central axis A, there is, for each turn, a position in which one of the baffles 110 extend from a bottom half of the inner surface to a horizontal plane H coinciding with the central axis A. In this position, at least, the baffle 110 divides the bottom half of the internal volume into a first compartment 121 and a second compartment 122. A compartment may thus be understood as a portion of the lower or bottom half of the interior volume of the reaction vessel 100 defined by the horizontal plane H passing through the central axis A, the inner surface of the reaction vessel 100, and the baffle 110. The volume of the first compartment 121 and the volume of the second compartment 122 may hence be understood to form the complete volume of the bottom half of the reaction vessel 110, i.e., half the total volume of the reaction vessel. As illustrated in the present figure, the distribution of the volume between the two compartments 121, 122 may be defined by the shape and position of the baffle 110 extending from the lower half of the inner surface to the horizontal plane H. Moving the baffle 110 to the left in FIG. 2a would result in the liquid volume of the first compartment 121 decreasing and the liquid volume of the second compartment increasing 122, and vice versa.

The present arrangement of the baffles 110 allows for a liquid, arranged in the first compartment 121, to reach the horizontal plane H without having to fill the entire bottom half of the vessel volume. The baffle 110 may hence act as a barrier separating the compartments 121, 122 from each other and preventing liquid from the first compartment 121 from leaking into the second compartment 122 (at least in the particular angular position shown in FIG. 2a). As the reaction vessel 100 is rotated, for instance clockwise, the liquid is allowed to pass over the edge 112 of the baffle 110 and into the second compartment 122, which eventually is filled. By continuing the rotation of the vessel 100 to a total rotation of 180°, the other one of the two baffles 110 is arranged such that it extends from the lower half of the inner surface up to the horizonal plane H, thereby dividing the bottom half of the interior volume into a first and second compartment 121, 122. Thus, what used to be the upper half of the interior volume of the vessel 100 is now the bottom half, and by rotating the reaction vessel, the entirety of the internal volume of the reaction vessel 100 is allowed to be wet by a volume of liquid which is smaller than half of the internal volume of the reaction vessel 100. This is for example beneficial when implemented in packed biofilm reactors, in which microbes are attached to a support frame, or packing material, that is evenly distributed across the whole internal volume of the reaction vessel 100. The packing material may be configured to act as a microbial attachment device or as a chemical catalyst promoting the conversion processes. By providing compartment-defining baffles 110 inside the vessel 100, a reduced volume of liquid (i.e., a volume less than half the interior volume of the vessel 100) is sufficient to wet the entire interior volume (or at least substantially all of the interior volume) of the vessel 100 during each revolution of the vessel 100.

By increasing the number of baffles 110, such as to three, four, five, or six baffles (or more), the size of the first compartment 121 and, accordingly, the amount of liquid needed to wet the entire first compartment 121, can be reduced. Reducing the amount of liquid may, in turn, allow for a reduced rotating mass and hence a reduced power consumption for rotating the vessel 100. It is presently envisaged that the number of baffles within a reaction vessel may suitably be from 2 to 10 (i.e., including 2, 3, 4, 5, 6, 7, 8, 9, or 10 baffles) to achieve the above-mentioned advantageous effects provided by the baffles. The amount of liquid needed to wet the entire interior volume of the vessel would normally not be reduced more by increasing the number of baffles to above 10 baffles. Also, the higher the number of baffles, the more weight the baffles would add to the reaction vessel, thereby requiring an increased power consumption for rotating the vessel. According to presently preferred embodiments, the number of baffles is 2, 3, 4, 5, or 6 baffles. Currently more preferred, the number of baffles is 3, 4, 5, or 6.

As mentioned above, the body of the reaction vessel 100 may not necessarily be limited to circular cross-sectional shapes. Other shapes are conceivable, such as cylinders with polygonal cross sections. FIG. 2b shows an example of such an embodiment, wherein the body of the reaction vessel 100 has a hexagonal cross section. Preferably, but not necessarily, the body of the vessel 100 may be rotationally symmetric with respect to the central axis A. In FIG. 2a, the circular cross section of the body of the vessel 100 is fully rotationally symmetric at any angle, and the degree of rotational symmetry of the complete vessel 100 is therefore determined by the distribution of the baffles 110 only. In FIG. 2b, the hexagonal shape of the body of the vessel 100 represents a 6-fold rotational symmetry, which is limited by the pair of baffles 110 to a 2-fold rotational symmetry.

Further, it will be appreciated that the top edge 112 of a baffle 110 may, in some embodiments and in some rotational positions of the vessel 100 extend not only to the horizontal plane H, but beyond the plane, as indicated in FIG. 2b.

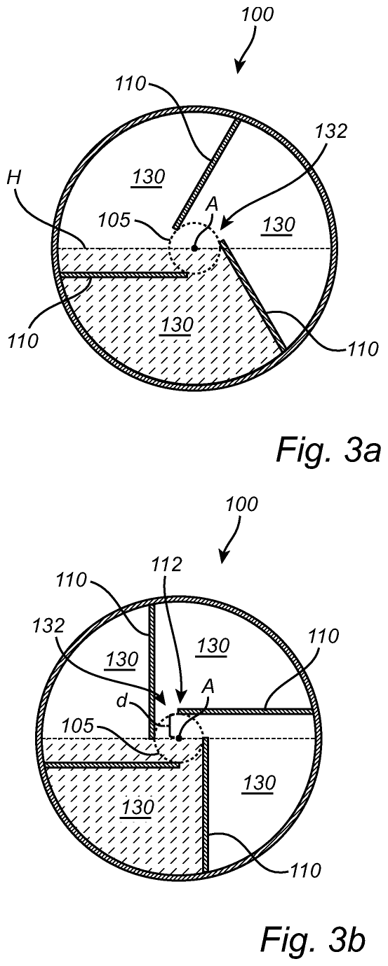

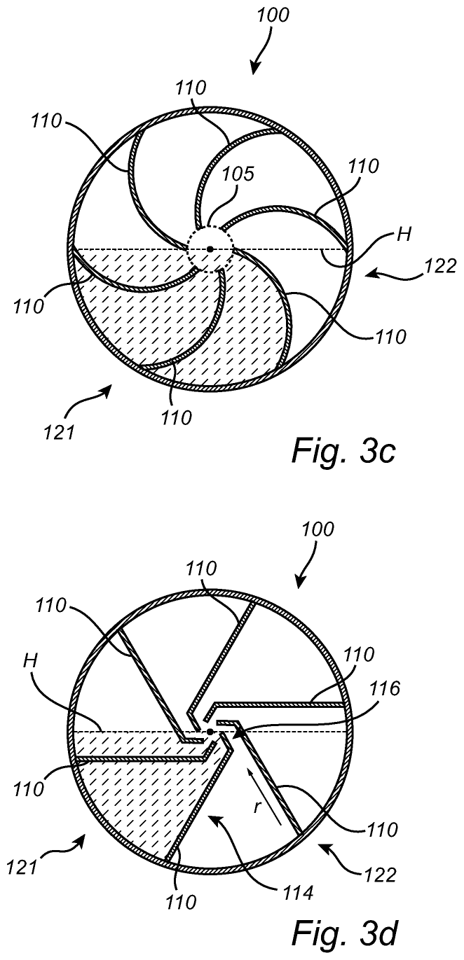

FIGS. 3a-d show embodiments of reaction vessels 100 which may be similar to the vessels 100 discussed above with reference to FIGS. 1a-b and 2a-b. However, the embodiments of the present figures differ in that FIG. 3a shows a configuration with three baffles 110 arranged about 120° apart along the cross-sectional circumference of the vessel body, FIG. 3b shows a variant with four baffles 110 about 90° apart, and FIGS. 3c and d show embodiments with six baffles 110.

In the embodiments shown in FIGS. 3a-d each of the baffles 110 extends from the inner surface of the vessel towards a central portion 105 for at least one angular position of the vessel 100. Further, the baffles 110 are oriented such that they define a plurality of chambers 130, wherein each chamber 130 is delimited by a portion of the inner surface of the vessel and two neighbouring baffles 110. The chambers 130 may be similarly sized, dividing the entire interior volume but the central portion into three chambers 130 (FIG. 3a), four chambers 130 (FIG. 3b), and six chambers (FIGS. 3c and d), respectively. Further, each pair of neighbouring baffles 110 defines an opening 132 between the chamber 130 and the central portion 105, allowing liquid to flow between the central portion 105 and each chamber 130.

For the purpose of the present disclosure, the term ‘compartment’ may be used to denote portions of the bottom half of the interior volume of the vessel 100 that are separated by a baffle 110. A compartment may hence be defined by a portion of the bottom half of the inner surface, a baffle and the horizontal plane H. The term ‘chamber’, on the other hand, may be used to denote a portion of the interior volume of the vessel 100 defined by a portion of the inner surface and two neighbouring baffles. The volume of a compartment 121 may hence exceed the volume of a chamber 130, as indicated e.g. in FIGS. 3a and b.

The baffles 110 may be arranged such that an edge 112 of each baffle is separated from the central axis A by a distance or spacing d, as shown in e.g. FIG. 3b. Put differently, each baffle 110 may be considered to be arranged at least slightly off centre with respect to the central axis A, such that each baffle extends along a direction r not intersecting the central axis, as shown in FIGS. 3a, b and d. The distance d may be understood as corresponding to a radius, or half the width, of the central portion 105 of the interior volume.

The baffles 110 may be substantially planar as shown in, for example, FIGS. 3a and b, or curved as shown in FIG. 3c. Other shapes than the one depicted are also possible, including S-shaped and folded cross sections. Further, the baffles may extend parallel to the central axis A or, in some examples, form a helix or spiral around the central axis A.

FIG. 3d shows an embodiment in which each baffle 110 comprises a main portion 114 extending in a direction r not intersecting the central axis A and a flow guiding edge portion 116 for guiding liquid medium contained in the reaction vessel 100. The flow guiding edge portion 116 may extend at an angle to the main portion 114 of the baffle 110 to guide the liquid towards the central portion 105 of the interior volume of the vessel 100. It is to be understood that said angle may be any angle within the range of from 0° to 180° to the main portion 114, as long as the flow guiding edge portion has the ability to guide liquid contained in the vessel. A person skilled in the art is able to choose a suitable angle depending on the number of baffles, as well as the shape and arrangement of baffles within the reaction vessel. The skilled person is further able to choose a suitable angle depending on which rotational speed is to be applied to the reaction vessel. The flow guiding edge portion 116 may be integrally formed with the main portion 114 of the baffle 110 or provided as a separate component that is attached to the main portion 114.

The reactions vessels 100 in FIGS. 2a-b and 3a-d are depicted in a position in which the at least one of the baffles 110 extends from the bottom half of the inner surface and at least to the horizontal plane H coinciding with the central axis A of the vessel 100. This may be understood as a momentary angular position of the vessel 100 as it rotates around the central axis A, and the vessel may either dwell in that position for a while or pass it in a continuous, rotational moment. In either case, the depicted positions of the vessel 100 allows the liquid to fill the first compartment 121 only, and as the vessel 100 continues to rotate allows the liquid to move into the second compartment 122 and to fill the second compartment 122 only. Hence, the present invention allows for the interior volume of the reaction vessel 100 to be compartmentalised in a way that allows for the liquid to sequentially move from one compartment to a neighbouring compartment during rotation of the reaction vessel 100.

As mentioned above, the top edge 112 of a baffle 110 may, in some embodiments and in some rotational positions of the vessel 100 extend not only to the horizontal plane H, but beyond the plane. This is indicated in FIG. 3a, where the baffle 110 on the right-hand side of the bottom half of the inner surface of the vessel is shown to extend beyond (i.e., above) the horizontal plane H (however, the top edge 112 is not indicated in FIG. 3a).

It is to be understood that for any number of baffles (≥2), for any arrangement of baffles, and for any cross-sectional shape of the reaction vessel, each baffle 110 has a theoretical maximum height, extending from the inner surface of the reaction vessel towards the interior of the reaction vessel. This maximum height can be calculated by a person skilled in the art based on the fact that the baffle shall divide a bottom half of the internal volume of the reaction vessel into compartments 121, 122 and that the plurality of baffles 110 shall be configured to allow a liquid medium contained within the reaction vessel to move sequentially from a first one of the compartments into a neighbouring one of the compartments during rotation of the reaction vessel. Accordingly, the height of a baffle must not be larger than to allow flow, preferably laminar flow (as defined by Reynolds number Re≤2300), from one compartment 121 to the next compartment 122. Further, for embodiments including at least three baffles 110, the maximum height of each baffle can be determined by the skilled person based on the fact that the baffles shall divide the interior of the reaction vessel into a plurality of substantially similarly sized chambers 130 and a central portion 105, and that each chamber 130 shall be defined by a portion of the inner surface and by two neighbouring baffles 110, and that said two neighbouring baffles shall define an opening 132 between said chamber 130 and the central portion 105 of the reaction vessel.

Furthermore, the width of the central portion 105 (i.e., twice the distance d as indicated in FIG. 3b) must not be smaller than to allow flow, preferably laminar flow (i.e., Reynolds number Re≤2300), from one chamber 130 to the next chamber 130. At the same time, it is to be understood that the larger the width of the central portion 105, the larger volume of liquid is required to fill a compartment 121 or a chamber 130, as seen for example by comparing FIG. 2a and FIG. 3b. The distance d from the baffle's edge 112 to the central axis A and the liquid volume, respectively, are both much larger in FIG. 2a than in FIG. 3b. Accordingly, it is preferable to arrange the edge 112 of each baffle as close to the central axis A as possible, i.e., the distance d should be as small as possible, while still allowing flow from one compartment or chamber to the next. It is to be understood that the distance d to be chosen is also related to the rotational speed to be applied to the reaction vessel during operation.

Further, the maximum radius of the inlet 101 and of the outlet 102 of the reaction vessel should be substantially similar to the distance d or the radius of the central portion 105, while the minimum radius of the inlet 101 and of the outlet 102 are only restricted by flow.

FIGS. 3a-3d show the maximum desirable liquid level, coinciding with the horizontal plane H of a reaction vessel, wherein the radius of the outlet 102 is substantially as large as the radius of the central portion 105.

Alternatively, according to other non-limiting embodiments, the reaction vessel may be operated at a lower liquid level than the maximum liquid level shown in FIGS. 3a-3d. Such a lower liquid level may be in between the lower edge of the central portion 105 and the horizontal plane H, may coincide with the lower edge of the central portion 105, or may be substantially lower than the lower edge of the central portion 105.

According to non-limiting embodiments of the present invention, the reaction vessel comprises three, four, or five planar baffles, respectively, wherein for each baffle, the reaction vessel is rotatable around the central axis A into a position in which the baffle extends from a bottom half of the inner surface to the horizontal plane H coinciding with the central axis. Each baffle extends from the inner surface in a direction r not intersecting the central axis.

According to an alternative non-limiting embodiment, the reaction vessel comprises six curved baffles, wherein for each baffle, the reaction vessel is rotatable around the central axis A into a position in which the baffle extends from a bottom half of the inner surface to the horizontal plane H coinciding with the central axis. The edge 112 of each baffle is arranged at a distance d from the central axis of the reaction vessel.

According to yet another non-limiting embodiment, the reaction vessel comprises six baffles, each baffle having a planar main portion 114 and a flow guiding edge portion 116 which is curved or angled compared to the direction r of the main portion 114, wherein for each baffle, the reaction vessel is rotatable around the central axis A into a position in which the baffle extends from a bottom half of the inner surface to the horizontal plane H coinciding with the central axis. The direction r is not intersecting the central axis.

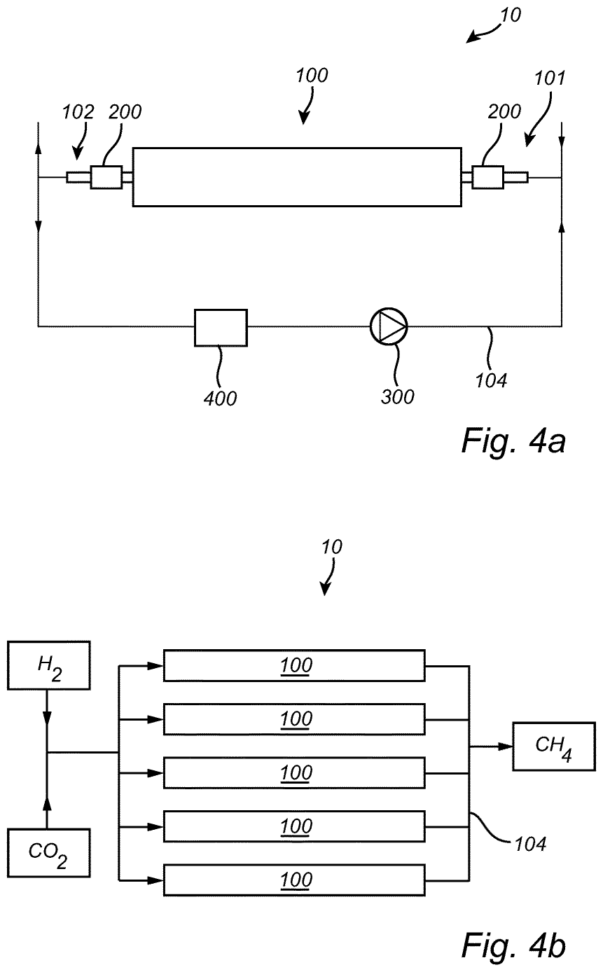

FIG. 4a shows a system 10 for converting a substrate to a product according to an example of the present invention, comprising a reaction vessel 100, and a rotational device 200. The reaction vessel 100 may be similarly configured as any of the vessels 100 discussed above with reference to the previous figures and is therefore not described in any further detail in the following.

The rotational device 200 may be configured to rotate the reaction vessel 100 around its central axis A to cause liquid contained within the reaction vessel to wet the interior of the reaction vessel 100, as previously discussed. The rotational device 200 may for example comprise a motor, such as an electric motor, and a gear system transmitting the movement from the motor to the vessel 100. The rotational device 200 may be included in a bearing supporting end portions of the vessel 100 as shown in FIG. 4a, or on any other part of the vessel such as an exterior surface of the main body of the vessel 100. The rotational force may in some examples be transmitted to the vessel by means of a wheel, a chain, or a belt (not shown), acting on the vessel 100.

Further, a conduit system 104 may be provided to allow a circular flow of liquid medium comprised in the system 10. The conduit system 104 may be arranged to provide a fluid connection between the outlet opening 102 and the inlet opening 101, in which case liquid medium may be recirculated within the system. Further, any unconverted substrate or reactants discharged via the outlet 102 could be returned to the inlet 101 via the conduit system 104. It will be appreciated that the conduit system 104 may comprise further outlets for discharging and/or separating liquid medium and/or product from the system.

According to the embodiment shown in FIG. 4a, the system 10 may further comprise a pump 300. Such a pump 300 may be provided to promote recirculation of liquid medium accommodated in the system 10.

The system 10 may further comprise a unit 400, according to the embodiment shown in FIG. 4a. The unit 400 may be employed to provide access to the circulating liquid medium. The access may for example be used for liquid level control, pH control, addition of nutrition(s) to the liquid medium, and/or sampling of liquid medium arranged in the system 10.

Generally, the presently disclosed system may suitably comprise means for measuring and controlling the temperature, pressure, exposure to light wavelength/intensity, pH, liquid flow rate, liquid recirculation rate, liquid composition, gas flow rate, gas composition, and/or rotational speed of the reaction vessel.

The system 10 may comprise one or several reaction vessels 100 as described above. Where the system comprises multiple reaction vessels, the vessels may be connected in parallel or in series, where gases and liquids from one vessel can be fed into another vessel. The conversion capacity of such a system can be adjusted by adding/removing one or more reaction vessels from the system.

The following non-limiting examples illustrate how a system comprising several reaction vessels connected in series/parallel may be beneficial. We assume a reactor (i.e., a reaction vessel) with a max. conversion capacity of (x) m³/min. If the reactor is using microbial biofilm to achieve conversion, any increase/reduction in the feeding gas rate will affect biofilm composition and formation, consequently changing the conversion capacity of the system itself.

By replacing this one reactor with a number of reaction vessels, the conversion can be reduced/increased by adding/removing one or more reaction vessels from the system. Secondly, one or more of the connected reaction vessels that form the system can be prepared and primed in a different facility and transported to the site to be used. This allows for more controlled conditions to be employed at a dedicated workshop for the construction and preparation (e.g. creation of biofilm) of the reaction vessels. Further, different operating conditions may be required in each vessel to enable a complete conversion and therefore a serial approach may be of benefit. Product accumulation in long vessels may reduce conversion efficiencies and therefore shorter length in parallel vessels may be preferred. FIG. 4b shows an embodiment in which five reaction vessels 100 are connected in parallel. In this specific and non-limiting example, the system is supplied with the gaseous substrates H₂ and CO₂ and is converting them into CH₄. Other combinations and reactions are however equally possible within the scope of the present invention, as described in more detail further below.

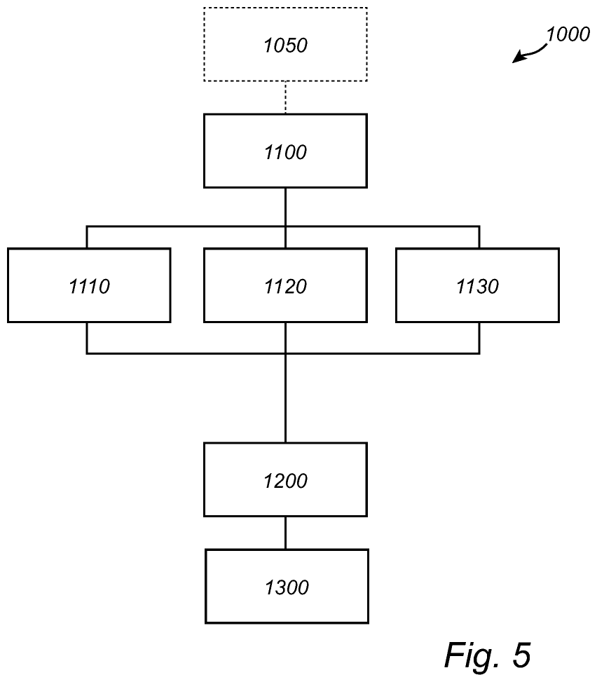

FIG. 5 schematically depicts a method 1000 for converting a substrate to a product, comprising operating a reaction vessel 100 or a system 10 as disclosed herein. More particularly, FIG. 5 depicts a method comprising adding 1100 a liquid medium and a substrate to the reaction vessel via the inlet of the reaction vessel, applying 1200 a rotational movement of the reaction vessel, to allow wetting of each of the compartments or chambers in sequence, thereby obtaining wetting of the entire interior volume of the reaction vessel by the liquid medium, and obtaining 1300 a product from an outlet of the reaction vessel, wherein the volume of liquid medium contained within the reaction vessel is smaller than half of the interior volume of the reaction vessel at each moment of converting the substrate to the product within the reaction vessel. Optionally, the method 1000 may further comprise adding 1050 a packing material to the reaction vessel before the liquid medium and/or the substrate is added 1100 to the reaction vessel. Further, it will be appreciated that according to some embodiments, the substrate may be converted to a product biologically, chemically, or biochemically. In case of a biological conversion, a microbial population may be added 1110 to the reaction vessel. In case of a chemical conversion, a chemical catalyst and/or chemical additive may be added 1120 to the reaction vessel. And for a biochemical conversion, the method may comprise adding 1130 a combination of a microbial population, and at least one of a chemical catalyst and a chemical additive to the reaction vessel.

The presently disclosed reaction vessel can be used to perform any conversions that are performed by previously known trickle bed reactors. These include both biological and chemical conversions, as well as combinations of biological and chemical conversions.

Non-limiting examples of substrates to add to the reaction vessel include:
(i) gaseous substrates, such as any one of the gases N₂, H₂, O₂, CO₂, CO, and CH₄, and any combination thereof,
(ii) non-gaseous substrates, such as sugars (e.g. glucose), alcohols (e.g. methanol), organic/non-organic acids (e.g. acetate, butyrate propionate, valerate/sulfuric acid), long chain hydrocarbons (e.g. benzene), carbonate salts (e.g. calcium carbonate), metals (e.g. zinc), polymers (e.g PLA, PHA), and any combination thereof, and
(iii) any combination of gaseous and non-gaseous substrates.

It is to be understood that some conversion processes require one or more co-substrates in addition to the main substrate(s), for the conversion to take place.

Non-limiting examples of products to obtain from the reaction vessel include:
(i) gaseous products, such as any one of H₂, CO₂, ammonia, certain alkanes (e.g., methane, ethane, propane, butane), and any combination thereof,
(ii) non-gaseous products, such as alcohols, aldehydes, carboxylic acids, olefins, polysaccharides, amino acids, flavonoids, polymers, solubilised salts, crystallised salts, and any combination thereof, and
(iii) any combination of gaseous and non-gaseous products.

According to a currently preferred embodiment, the substrate is a gaseous substrate, and the product is a gaseous product. Currently more preferred, the gaseous substrate is a combination of hydrogen (H₂) and carbon dioxide (CO₂), and the gaseous product is methane (CH₄).

It is to be understood that for biological conversions, either pure or mixed microbial populations chosen from bacteria, archaea, and algae, may be used, as required by the circumstances of any specific biological conversion process.

For biological conversions performed in the presently disclosed reaction vessel, the reaction vessel as such may serve as a biofilm reactor which, thanks to the baffles, has a high surface area of contact between the microbes and the liquid medium including the substrate. This scenario is relevant where no gas-mass transfer is needed, as well as where gas-mass transfer is needed.

To enhance the conversion of substrate to product, a packing material can be added to the reaction vessel. In the case of biological conversions, the microbes can additionally get attached to the surface of the packing material to form biofilms, or the microbes can simply be in suspension in the liquid media. In both cases, the packing material helps with increasing the surface area of contact between the gas and the liquid, consequently increasing the gas-mass transfer to the liquid.

Non-limiting examples of biological conversions by microbes (wild strains or genetically modified) that can be performed by use of the presently disclosed reaction vessel include:
1. Conversion of H₂ and CO₂ to carboxylic acids and/or CH₄ by bacteria and/or archaea, (pure and mixed cultures), e.g. acetobacterium, clostridium, methanobacterium and/or methanococcus spp.
2. Production of hydrogen from sugars (e.g. sucrose) with e.g. Thermotogae or Clostridium spp.
3. Aerobic/anaerobic treatment of organic or inorganic polluted wastewaters and sludges or slurries.
4. Aerobic conversion of styrene to(S)-Styrene oxide with Pseudomonas sp. followed by production of phenethyl alcohol by hydrogenation.
5. Oxidization of hydrogen sulfide (H₂S) to form sulfuric acid by Acidithiobacillus spp.
6. Production of antibiotics (e.g. Cephalosporin C by Cephalosporium acremonium). The fungi in solution need high-rate oxygenation.
7. Production of ammonia and hydrogen by nitrogen fixing microbes.
8. Conversion of CO₂ and organic acids to H₂ by bacteria including photosynthetic bacteria e.g. Alphaproteobacteria, Clostridium spp.
9. Conversion of CO₂, H₂, CH₄ and carboxylic acids to polyhydroxyalkanoates e.g. Alphaproteobacteria, Betaproteobacteria spp.
10. Microbial production of acetone, methanol, ethanol, propanol, butanol and pentanol from CO, CO₂, CH₄, H₂ or from mixtures of gases, such as syngas (i.e., a mixture of CO and H₂), via for example Clostridia and Proteobacteria sp. including methanotrophs.
11. Microbial conversion of fluorinated pollutant compounds such as chlorofluorocarbons, for example by Proteobacteria, Actinobacteria, Archaeal sp.

For chemical conversions performed in the presently disclosed reaction vessel, the chemical reaction may take place in the absence or presence of a catalyst. Further, it may be carried out in the presence or absence of a packing material in the reaction vessel. If there is no need for catalyst, adding a packing material can perform the function of increasing the surface area of contact between the reactants. Alternatively, the packing material can be one or more of the reactants, or the packing material can be inert but coated with one or more of the reactants. On the other hand, if there is need for a catalyst to perform the chemical conversion, the packing material can be the catalyst, or the packing material can be coated with the catalyst.

Non-limiting examples of chemical conversions that can be carried out by use of the presently disclosed reaction vessel include:
1. A two-step process for the production of hydrogen peroxide; first, a process where the catalyst is coating the packing material, followed by a process without a catalyst. Two reaction vessels coupled in series may then be used. The product of the first one is fed to the next one for the second reaction:
   a) Hydrogenation of 2-Ethylanthraquinone (EAQ) to 2-ethylanthrahydroquinone (EAQH₂) over a Pd/Al₂O₃ catalyst.
   b) Oxygenation of EAQH₂ to hydrogen peroxide without catalyst.
2. Enhanced weathering for CO₂ capture. Alkaline minerals (e.g., Calcite, Basalt)+water+CO₂→Carbonate minerals. In this case, a packing material can be the alkaline mineral.
3. Refining of oil and gas, for example, hydrodesulphurization of naphtha. In this case, a packing material is coated with a catalyst (e.g. platinum, rhenium, molybdenum), the liquid is the unrefined oil and the gas is hydrogen. Hydrogenation of the sulphur occurs over the catalyst, and it gets removed as H₂S.
4. Ammonia removal by wet oxidation. Ammonia is removed from industrial or biological effluents by reacting with oxygen over a catalyst.

A packing material added to the reaction vessel may for example be a porous material, providing a large surface of contact with the substrate as well as with any microbial population or chemical catalyst involved in the conversion process.

The presently disclosed reaction vessel may be made of a transparent material or a non-transparent material or a combination thereof. Non-limiting examples of transparent materials suitable in this context are glass and plastic. Non-limiting examples of suitable non-transparent materials are metal and plastic. The baffles provided within the reaction vessel may be made of the same type of material as the main body of the reaction vessel or may be made of a different material. More particularly, the baffles may suitably be made of metal and/or plastic.

The presently disclosed method may be performed under dark or light conditions, or in other words, in the presence or absence of light. In cases where a conversion process would be improved by being exposed to light, the reaction vessel, or a part thereof (such as one or more, but not all, compartments or chambers of the reaction vessel), is advantageously made of a transparent material; at least the outer parts of the reaction vessel through which the light would need to penetrate to achieve the desired effect. Alternatively, the reaction vessel may comprise a source of light (e.g., a light emitting diode, LED) internally.

As mentioned above, the flow of liquid from one compartment or chamber to the next within the reaction vessel is influenced by the relative dimensions and arrangements of baffles and the central portion of the vessel. However, flow is also affected by the speed of rotation. It is to be understood that a person skilled in the art can calculate a range of suitable rotational speeds applicable for different conversion processes. Further, the rotational movement can be applied to the reaction vessel continuously, intermittently, or stepwise, or in any combination thereof, depending on the type of conversion which is to take place. By applying rotational movement, liquid medium disposed with the reaction vessel will periodically immerse each compartment or chamber of the reaction vessel. While one part of the interior of the vessel is wetted, the other parts will temporarily be non-wetted. It is to be understood that the skilled person can determine suitable lengths of wetting time and non-wetting time in order for a conversion process to run efficiently.

The presently disclosed reaction vessel and system may be operated under a range of pressures and/or a range of temperatures, and the herein disclosed method may be performed under various pressures and/or temperatures, as required by the specific circumstances of each conversion process. Accordingly, the presently disclosed reaction vessel may be made of a material which is durable and operates safely under various pressures and/or temperatures. It is to be understood that a person skilled in the art is able to choose a material suitable for the conditions required for any specific conversion process.

### Experimental Section

### Materials and Methods

### Reactor Configuration

### Rotating Compartmented Biofilm Reactor System (RCBRS) According to the Present Invention:

The prototype consisted of three main parts: a) the main body, b) the baffles and packing media and c) the support and rotation mechanism.
a) The main body was constructed from clear PVC tube with welded PVC end caps that supported 2 rotary unions (Festo Ltd, USA), one on each side (Figure A).
   The dimensions of the PVC tube were, Length: L=0.8 m, radius: r=0.05 m with a total inner volume: V=6.3 L, a total free volume (inner space not occupied by baffles and packing material) VF=4.9 L and liquid volume VL=1 L. The two caps sealed the tube on each side with the help of rubber gaskets so that fluids could only enter/exit the reactor through the rotary unions. The rotary unions had a 0.008 m bore and were connected to nylon tubing of the same diameter for the transferring of fluids.
b) The baffles were constructed from an acrylic sheet that was cut into 4 pieces of equal dimensions: Length: L=0.8 m, Width: W: 0.05 m and Thickness: T: 0.003 m. The 4 pieces were fixed in place inside the PVC tube so that they created an arrangement of baffles as illustrated in FIG. 3b with a common centre (105) radius of approximately 0.002 m. The reactor was packed with support media for microbial attachment (Kaldnes K1).
c) The reactor was supported in horizontal position by 4 castors that were fixed on a metal frame. A 12 VDC motor was attached to a rubber wheel that was in contact with the main body of the reactor thus providing its rotation. The motor was controlled manually through a potentiometer that allowed a rotational speed range of 0-5 rpm.

The temperature of the culture was regulated by placing the whole reactor unit inside an incubator.

### Continuously Stirred Tank Reactor (CSTR):

The reactor consisted of a glass cylinder with dimensions L=0.22 m, r=0.05 m, with a total inner volume V=1.73 L and liquid volume VL=1 L. The cylinder was sealed with rubber caps on both sides and was equipped with several ports for gas feeding/sampling and measurement of pH and temperature (Figure B). Mixing was achieved with a Rushton type impeller with six blades and diameter D=0.06 m. Motion of the impeller was achieved by means of magnetic coupling to a 12 VDC motor situated underneath the reactor. The impeller speed could be controlled within a range of 0-2500 rpm. Four vertical rods (0.008 in diameter) extending from the top to the bottom of the vessel, were placed diametrically close to the walls. These acted as baffles in order to create turbulence during mixing. The temperature of the culture was continuously monitored and regulated by means of a heating pad and a thermocouple connected to a temperature controller.

### Gas Supply/Measurement/Pressure

The feeding gas was supplied by gas cylinders (Air Liquide) and gas flow was controlled with mass-flow controllers (Alicat MC series, Alicat Scientific, USA), individually calibrated for each gas. Analysis of the product gas was performed with infrared gas analysers (Boxed GasCard, Edinburg Instruments, UK). Pressure inside the reactor was controlled with the help of a back-pressure valve (Fluidcontrols Ltd, UK).

### Culture

The starting culture used in both reactors was mesophilic digested sewage sludge, collected from a local anaerobic digestion wastewater treatment facility. The sludge was filtered through a 300 μm sieve before inoculation. The temperature for all experiments was 37±0.5° C.

### Experiments

Start up to steady state comparison of RCBRS and CSTR-Gas input rate 25 ml/min, gas ratio 4:1 H₂:CO₂.

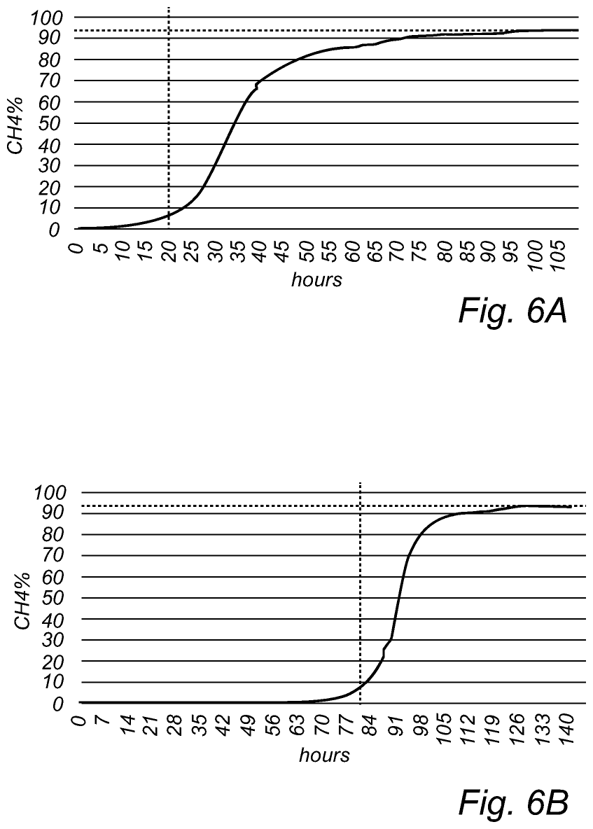

FIG. 6a shows the evolution in methane production from start-up to steady state in the RCBRS and FIG. 6b shows the evolution in methane production from start-up to steady state in the CSTR. Since during the initial growth phase, methane production and growth are coupled, it can be reasoned that methanogens in the RCBRS entered the exponential growth phase much faster (around 20 h) than in the CSTR (around 80 h). It can also be seen that at steady state, both systems peaked at around 93% CH₄ in the product gas.

The reasoning behind the outcome is as follows: In the CSTR, 1 L of liquid culture occupies exactly 1 L of space in the reactor. In order to diffuse the gas into the liquid, high intensity mixing is required (1500 rpm) which could cause partial inhibition through the shear forces created from the impeller and the baffles. In the RCBRS, this 1 L of liquid gets spread over the packing media and occupies up to 4.9 L of space. More particularly, the rotation of the reactor, causing the 1 L of liquid to move from one compartment to the next, results in sufficient moisture for at least 4.9 L of reactor volume. The result is a much higher surface area of contact between the gas and the liquid phase which seems to increase gas mass transfer dramatically.

The effect seems to get affected by the speed of rotation which determines how long the wetted parts get exposed to the gas phase. As the reaction vessel of the RCBRS rotates, the parts that emerge from the liquid phase into the gas phase carry with them a portion of the liquid media which creates a layer on the surface of the packing material. This layer gets thinner the more time it is exposed, as the liquid slowly descends back to the bottom of the reactor.

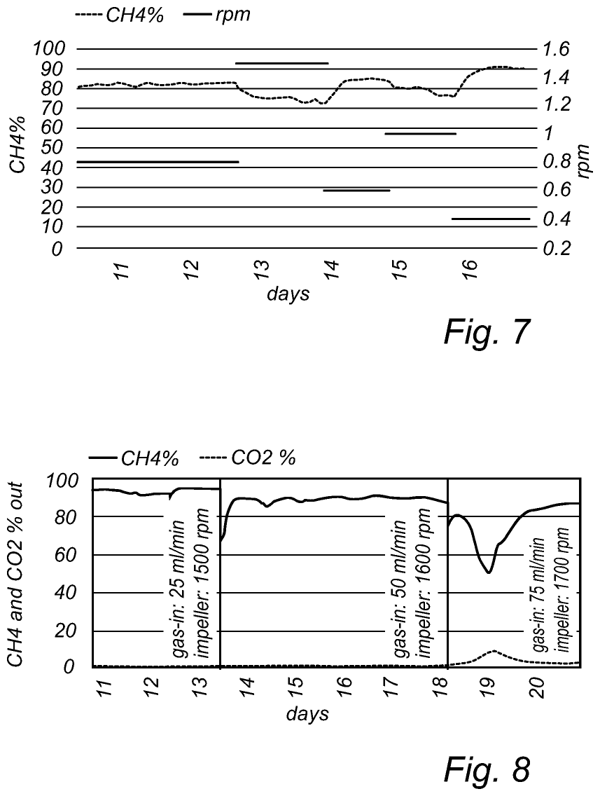

FIG. 7 illustrates how methane formation rate increases/decreases relative to the rotational speed of the reaction vessel of the RCBRS as presently disclosed. An advantage of the RCBRS design is that methanation capacity increases as the energy input for the rotation of the device decreases, which is the exact opposite of what occurs with reactors that rely on intense agitation. In the previously known CSTR design, in order to increase solubilisation of gases, the impeller speed needs to increase thus increasing the required energy input, as shown in FIG. 8.

FIG. 8 shows methane production in the previously known CSTR at three different gas feeding rates 25, 50 and 100 ml/min and at 3 different impeller speeds 1500, 1600 and 1700 rpm. It can be seen that every time there is an increase in the gas feeding rate, this needs to be followed by an increase in agitation in order for methane production to be stable.

In the case of the RCBRS according to the present invention, when there is an increase in gas feeding rates, methane production rate stability can be achieved by reducing the rotation frequency of the reactor.

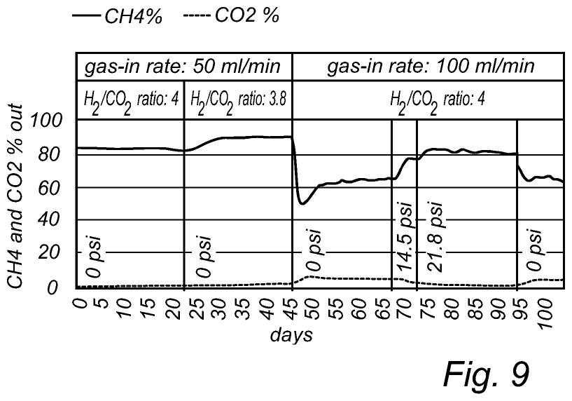

The internal pressure of the reaction vessel of the RCBRS is another parameter that can be used to enhance gas solubilisation when needed. In FIG. 9, pressure changes from 0 to 14.5 and 21.8 psi can be seen to help increase methane production efficiency when it falls due to doubling of the gas feeding rate from 50 to 100 ml/min. It is of interest that when the pressure drops back ambient (from 21.8 to 0 psi) production efficiency returns to the same levels as before the pressure increase. This is encouraging as it shows no noticeable inhibitory effect to the microbes due to the pressure drop.

The results presented here for both reactors are from a period of 20 days from start-up and therefore do not include methanation experiments with a mature culture. In the previously known CSTR, methanation rates can increase dramatically after a month of operation as the numbers of hydrogenotrophic methanogens tend to go up with a careful combination of increasing gas-feeding rates, increasing impeller speeds and the periodic washout of inhibitory compounds and the addition of nutrients.

The same is true for the RCBRS according to the present invention, as the experiments presented here did not include the creation of biofilms. Methanogenic biofilms need periods of at least 1-2 months to evolve and mature, and the presented methane production was due to microbes in suspension. This very fact, however, indicates the capacity of the herein disclosed design to achieve high gas mass transfer rates, similar to the ones that could be achieved with intense agitation, but at a fraction of the power needed (35 W for the CSTR at 1500 rpm vs. 1 W for the RCBRS at 1 rpm) as measured by a power meter (brennenstuhl PM231). The creation and maturation of methanogenic biofilms can be safely expected to further increase methanation rates and efficiency as shown in various previous studies as referred to elsewhere herein.

### Conversion Efficiency of the RCBRS

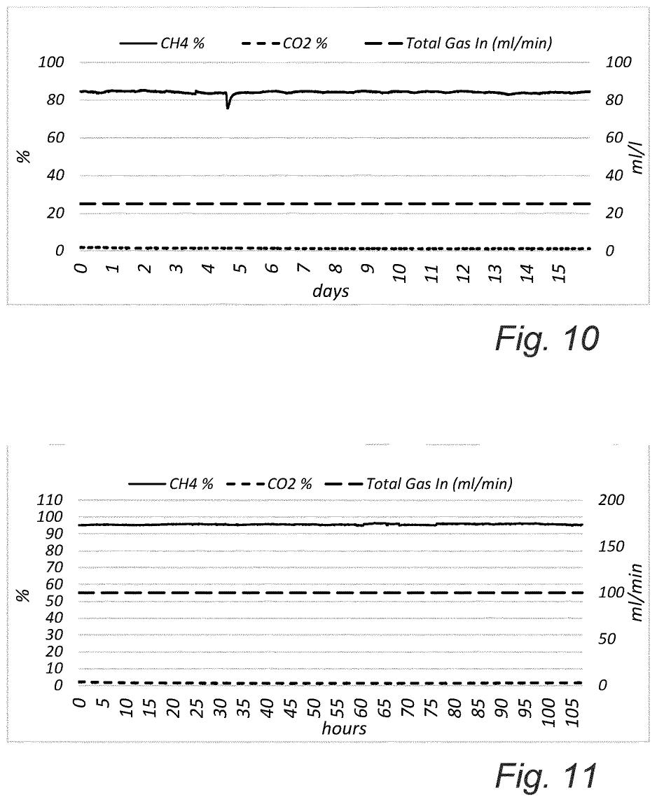

FIGS. 10 and 11 show that the RCBRS creates the right conditions to produce methane from H₂ and CO₂ gases very consistently.

FIG. 10 shows stable conversion without any further addition of nutrients after one month from start-up of the reactor, in the phase during which biofilm was growing but had not reached maturity and was not yet visible. Conversion efficiency (%) is shown on the left-hand y-axis.

Total gas in was 25 ml/min (right-hand y-axis). Rotation of the reaction vessel was 0.6 rpm. FIG. 11 demonstrates stable conversion at higher inflow of gas (100 ml/min) after two months of operation, when biofilm formation was visible. Rotation of the reaction vessel was 0.8 rpm.

FIG. 12 shows the effect of pressure on conversion efficiency once the biofilm had reached maturity. An increase from 0 to 20 psi resulted in a 60% increase in conversion efficiency. An increase from 0 to 30 psi resulted in a 68% increase in conversion efficiency. FIG. 12 illustrates that pressure in the RCBRS can be utilised to increase conversion rates without any negative effects for the microbial culture.

### Production of Intermediate Metabolites, Comparison of RCBRS and CSTR

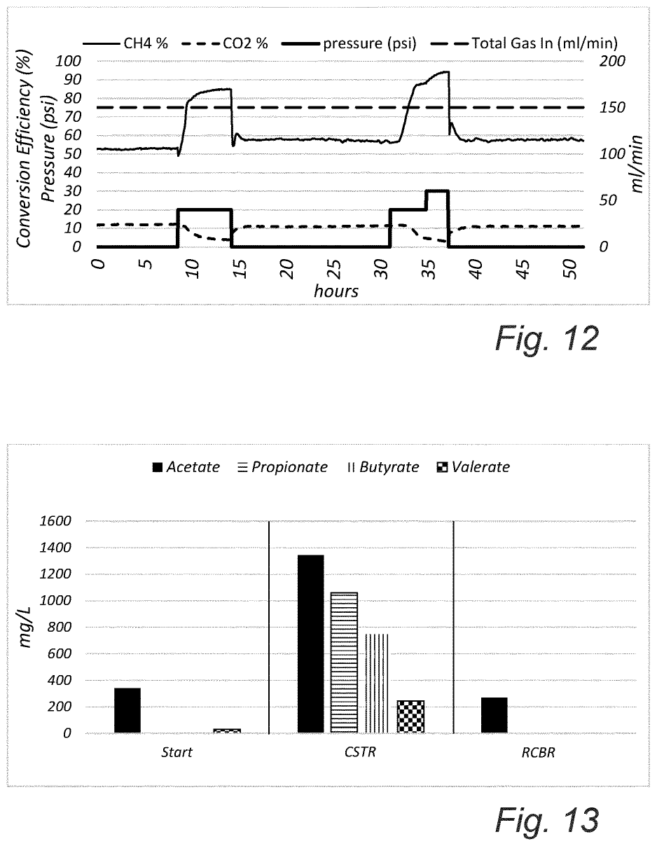

FIG. 13 shows the difference in microbial culture intermediate metabolites, i.e., volatile fatty acids (VFAs), produced and accumulated on a CSTR and on the RCBRS after one month of operation. Reactor operations targeted the production of biomethane and not VFAs in this case. Acid accumulation occurs when non-methanogenic microbial groups compete with methanogens for a share of the H₂/CO₂ feed and typically results in a sub-optimal biomethanation production system. Both reactors were inoculated with the same culture, which had an initial VFAs profile as indicated in FIG. 13 (‘start’). After one month, the total VFAs level in the CSTR had increased from 341 to 3395 mg/L, which indicated a loss of H₂/CO₂ through the homoacetogenic route and a disturbance in the conversion of heavier acids (propionic/butyric/valeric) to acetate. In contrast, the level of VFAs in the RCBRS remained unchanged (slight drop from 341 to 271 mg/L), which indicates a reduction in homoacetogenesis. Both reactors operated at 37° C., with a total liquid volume of 1 L and a constant gas feeding rate of 25 ml/min of H₂/CO₂ mix at a ratio of 4/1 v/v.

The above embodiments are to be understood as illustrative examples of the invention. Further embodiments of the invention are envisaged. It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

### REFERENCES

Garcia-Ochoa, F., Gomez, E., 2009. Bioreactor scale-up and oxygen transfer rate in microbial processes: An overview. Biotechnol Adv 27, 153-176. https://doi.org/10.1016/j.biotechadv.2008.10.006
Garcia-Ochoa, F., Gomez, E., 2004. Theoretical prediction of gas-liquid mass transfer coefficient, specific area and hold-up in sparged stirred tanks. Chem Eng Sci 59, 2489-2501. https://doi.org/10.1016/j.ces.2004.02.009
Gill, N. K., Appleton, M., Baganz, F., Lye, G. J., 2008. Quantification of power consumption and oxygen transfer characteristics of a stirred miniature bioreactor for predictive fermentation scale-up. Biotechnol Bioeng 100, 1144-1155. https://doi.org/10.1002/bit.21852
Karimi, A., Golbabaei, F., Mehrnia, M., Neghab, M., Mohammad, K., Nikpey, A., Pourmand, M., 2013. Oxygen mass transfer in a stirred tank bioreactor using different impeller configurations for environmental purposes. Iranian J Environ Health Sci Eng 10, 6. https://doi.org/10.1186/1735-2746-10-6
Mocciaro, C., Martínez, O. M., Barreto, G. F., 2011. Assessment of mass transfer in the stagnant liquid regions in trickle-bed reactors. Chemical Engineering Journal 173, 813-827. https://doi.org/10.1016/j.cej.2011.08.032
Patterson, T., Savvas, S., Chong, A., Law, I., Dinsdale, R., Esteves, S., 2017. Integration of Power to Methane in a Waste Water Treatment Plant-A Feasibility Study. Bioresour Technol 245, 1049-1057. https://doi.org/10.1016/j.biortech.2017.09.048
Rusmanis, D., O'Shea, R., Wall, D. M., Murphy, J. D., 2019. Biological hydrogen methanation systems—an overview of design and efficiency. Bioengineered 10, 604-634. https://doi.org/10.1080/21655979.2019.1684607
Savvas, S., Donnelly, J., Patterson, T., Chong, Z. S., Esteves, S. R., 2017. Biological methanation of CO₂ in a novel biofilm plug-flow reactor: A high rate and low parasitic energy process. Appl Energy 202, 238-247. https://doi.org/10.1016/j.apenergy.2017.05.134
Strübing, D., Moeller, A. B., Möβnang, B., Lebuhn, M., Drewes, J. E., Koch, K., 2018. Anaerobic thermophilic trickle bed reactor as a promising technology for flexible and demand-oriented H2/CO2biomethanation. Appl Energy 232, 543-554. https://doi.org/10.1016/j.apenergy.2018.09.225
Wik, T., 2003. Trickling Filters and biofilm reactor modelling. Rev Environ Sci Biotechnol. https://doi.org/10.1023/B

What is claimed is:

1. A reaction vessel for converting a substrate to a product, comprising:
an inlet opening for introducing a liquid medium and/or a substrate within said reaction vessel,
an outlet opening for obtaining a product from said reaction vessel, a length direction extending substantially along a central axis (A) of said reaction vessel, and
a plurality of baffles distributed along a circumference of an inner surface of the reaction vessel, wherein each baffle extends along said length direction and from an inner surface of the reaction vessel towards an interior of the reaction vessel,
wherein, for each baffle, the reaction vessel is rotatable around the central axis into a position in which the baffle extends from a bottom half of the inner surface at least to a horizontal plane (H) coinciding with the central axis, thereby dividing a bottom half of the internal volume of the reaction vessel into compartments,
wherein the plurality of baffles are configured to allow a liquid medium contained within the reaction vessel to move sequentially from a first one of the compartments into a neighbouring one of the compartments during rotation of the reaction vessel.

2. The reaction vessel according to claim 1, wherein each baffle has a length extending along the length direction of the reaction vessel and an edge extending along said length, wherein the edge is arranged at a distance (d) from the central axis of the reaction vessel.

3. The reaction vessel according to claim 1, wherein at least a main portion of each baffle extends from the inner surface in a direction (r) not intersecting the central axis.

4. The reaction vessel according to claim 1, wherein each baffle comprises a flow guiding edge portion configured to guide liquid medium contained in the reaction vessel.

5. The reaction vessel according to claim 4, wherein, for each baffle, the flow guiding edge portion extends in a direction at an angle to the direction in which the main portion of the baffle extends from the inner surface.

6. The reaction vessel according to claim 1, wherein the plurality of baffles comprise at least three baffles, which baffles divide the interior of the reaction vessel into a plurality of substantially similarly sized chambers and a central portion, and wherein each chamber is defined by a portion of the inner surface and by two neighbouring baffles, wherein said two neighbouring baffles define an opening between said chamber and the central portion of the reaction vessel.

7. The reaction vessel according to claim 6, wherein said at least three baffles are substantially evenly distributed within said reaction vessel.

8. The reaction vessel according to claim 1, wherein said inlet opening and said outlet opening are located at or near opposite ends of said reaction vessel.

9. (canceled)

10. The reaction vessel according to claim 1, wherein the reaction vessel is for converting a gaseous substrate to a product, such as a gaseous product.

11. A system for converting a substrate to a product, comprising:
a reaction vessel according to any one of the preceding claims, and
a rotational device for rotating said reaction vessel around the central axis of the reaction vessel.

12. The system according to claim 11, wherein the outlet opening is in fluid connection with the inlet opening to allow a circular flow of liquid medium contained within the system, optionally wherein the system further comprises a pump configured to recirculate liquid medium contained within the system.

13. The system according to claim 11, further comprising a unit configured to perform liquid level control, pH control, addition of nutrient(s) to a liquid medium contained within the system, and/or sampling of liquid medium contained within the system.

14. The system according to claim 11, wherein when the system is operated, the reaction vessel further comprises a packing material, optionally wherein said packing material is configured to act as a microbial attachment device or as a chemical catalyst.

15. The system according to claim 11, comprising at least two reaction vessels connected in series or in parallel.

16. A method for converting a substrate to a product, comprising operating a reaction vessel according to claim 1.

17. The method according to claim 16, comprising adding a liquid medium and a substrate to the reaction vessel via the inlet of the reaction vessel, applying a rotational movement of the reaction vessel to allow wetting of the entire interior volume of the reaction vessel by the liquid medium, and obtaining a product from an outlet of the reaction vessel, wherein the volume of liquid medium contained within the reaction vessel is smaller than half of the interior volume of the reaction vessel at each moment of converting the substrate to the product within the reaction vessel.

18. The method according to claim 17, wherein the rotational movement is continuous, intermittent, stepwise, or any combination thereof.

19. The method according to claim 16, further comprising adding a packing material to the reaction vessel before adding the liquid medium and/or the substrate to the reaction vessel.

20. The method according to claim 16, comprising:
(i) converting the substrate to a product biologically, wherein the method further comprises adding a microbial population to the reaction vessel via an inlet of the reaction vessel, optionally wherein the microbial population can form a biofilm within the reaction vessel;
(ii) converting the substrate to a product chemically, wherein the method optionally further comprises adding a chemical catalyst and/or a chemical additive to the reaction vessel via an inlet of the reaction vessel; or
(iii) converting the substrate to a product biochemically, wherein the method further comprises adding a microbial population, a chemical catalyst, and/or a chemical additive, to the reaction vessel via an inlet of the reaction vessel.

21. (canceled)

22. (canceled)

23. The method according to claim 16, wherein the substrate is gaseous, optionally wherein the product is gaseous, and wherein the substrates are hydrogen and carbon dioxide, and the product is methane.

24. (canceled)
